(19) United States
(12) Patent Application Publication
(10) Pub. No.: US 2018/0344977 A1
(43) Pub. Date: Dec. 6, 2018

(54) PNEUMATIC DEVICE FOR HOLDING AND MOVING AN ELONGATE OBJECT, AND MEDICAL SYSTEM INCORPORATING SUCH A DEVICE

(71) Applicants: UNIVERSITY DE STRASBOURG (Etablissement Public National à Caractère Scientifique, Culturel et Pro, STRASBOURG (FR); CENTRE NATIONAL DE LA RECHERCHE SCIENTIFIQUE (Etablissement Public National à Caractère Scienti, PARIS (FR); INSTITUT HOSPITALO-UNIVERSITAIRE DE CHIRURGIE MINI-INVASIVE GUIDEE PAR L'IMAGE (Fondation de Coo, STRASBOURG (FR); INSTITUT NATIONAL DES SCIENCES APPLIQUEES (Etablissement Public National à Caractère Scient, STRASBOURG (FR)

(72) Inventors: Mathieu NIERENBERGER, STRASBOURG (FR); Laurent BARBÉ, CHATENOIS (FR); Pierre RENAUD, SCHILTIGHEIM (FR); Benoît WACH, STRASBOURG (FR); Arnaud BRUYAS, STRASBOURG (FR)

(21) Appl. No.: 15/778,642

(22) PCT Filed: Nov. 24, 2016
(86) PCT No.: PCT/FR2016/053078

§ 371 Date: May 24, 2018

(30) Foreign Application Priority Data
Nov. 25, 2015 (FR) 1561373

(51) Int. Cl. A61M 25/01; A61B 1/00

(52) U.S. Cl. CPC A61M 25/0113; A61B 2017/00544; A61B 1/00147; A61M 25/0116

(57) ABSTRACT

A pneumatic device for holding and moving an elongate object, includes an annular hollow body through which the object passes and which is composed of two end parts forming jaws and a median segment connecting the two jaws to each other, with ease of movement of one with respect to the other under the effect of a controlled deformation of at least one portion of the median segment, where the various parts of the hollow body define respective chambers, the jaws have elastically deformable internal membranes, in the chambers of the two end parts are in fluidic communication with the chamber of the median segment, in each case by way of at least one respective calibrated flow element, the injection of pressurized gaseous fluid being effected by way of a single feed line.

This invention relates to the field of assisted—and even automated—manipulating of instruments or tools, or more generally elongated pieces or objects, in particular in applications that require high precision and maximum safety, such as that of surgical interventions on living subjects or actions that take place close to a man, and even in interaction with him.

In these contexts, the invention has as its object a pneumatic device for holding and moving an elongated object, a medical or industrial system that integrates at least one such device, and a method for inserting an elongated object via such a system.

In numerous technical fields, there is a need for automatic or semi-automatic devices that make it possible to move an elongated element or piece gradually, in particular for the purpose of inserting it into a body or another element.

This is true in particular for the medical field and more specifically surgery.

Already, a substantial number of robotic devices are being proposed for assistance with medical and surgical motions. For many of them, the robotic system is designed for the manipulation of tools such as surgical instruments, in particular in the form of elongated bodies, or needles in the case of, for example, interventional imaging.

In minimally invasive surgery, the fact that they traverse trocars determines the shapes and designs of tools and instruments that are used and limits the number of independent movements that can be performed. The translational movement along the axes of the tools, of oblong geometry, is then very heavily stressed.

In the same way, percutaneous procedures, which consist in inserting needles or analogous medical tools for getting into an area of interest, require a translational movement of needles of broad amplitude, which movement has to be carried out with a very high level of safety.

Within all of these contexts, if the oblong and elongated geometry of the objects that are to be moved is a constant, the diameters of their generally cylindrical bodies can widely vary, even during the same motion. It is the same with their length.

Most of the existing solutions are based on actuating systems that are integral with the tool that entails a modification of the standard tool (which is not desirable). The translational movement is generally carried out via a transformation of movement (passage from a rotational movement to a translational movement in particular). If need be (breakdown, medical emergency), the decoupling between the tool and the feed system is quite complex and can involve the safety of the patient. Furthermore, the increasingly widespread use of imagers (echography, fluoroscopy, X scanner, MRI) when the motion is being carried out makes it difficult and even impossible to employ a number of actuating technologies that are currently in use.

Various known techniques for assisted movement, motorized or mechanized, of the step-by-step or sequential type apply the so-called kinematic principles of the caterpillar (“inchworm” in English), in particular with regard to the components that can be inflated or actuated pneumatically.

Various implementations of these principles have already been proposed in the medical and surgical field, either to allow the at least partially automated advancement of an elongated object (for example, an endoscope) in a subject's natural conduit (see, for example: US 2010/022947; EP 1 792 561; U.S. Pat. No. 5,398,670; WO 02/068035), or to move, from the outside and in a partially automated way, at least one elongated object that has to be inserted into the body of a subject (see, for example: WO 2014/068563).

However, these known embodiments using an “inchworm”-type kinematics have at least some of the drawbacks below: complex structure; significant axial bulk; multiple constituent components; incompatibility with certain medical imaging technologies; limited adaptation to objects that have variable diameters or different values; use of various types of energy.

In addition, these embodiments all have the common drawback, on the one hand, of requiring several, i.e., at least two, feed lines for the various active components of the moving devices, on the other hand, of having to guide the various lines in a synchronized manner (difficult to accomplish when said lines are of significant lengths), and, finally, of having limited ability to adapt how said active components of the device engage with the elongated object that is to be moved.

Another field in which there is a growing need for preferably automated or at least semi-automated devices is that of the operating contexts and environments in which a motorized or actuated device is to operate in interaction with a human operator, by imitating or by assisting the motion of such an operator or else the environments with tight constraints, excluding in particular the use of electromechanical, electrotechnical or electronic technologies.

Thus, it is possible to cite the case of tasks of assembling mechanical components on assembly lines, where so-called collaborative robots for assisting the operator are now being proposed. For these devices, weight is a risk factor in the event of a collision. The existence of inherent flexibility to which one skilled in the art refers as compliance is a property that is furthermore important for managing the robot/operator contact. Finally, it is necessary to propose robots of minimum size so as not to hamper the operator in his movements and his collaboration with the robot.

In an industrial setting, it is also possible to cite automated tasks of the so-called “peg-in-hole” type (“plug in hole” or “plug-in” hole) in which the motions of assembling mechanical components are carried out. The assemblies are often based on centering elements for referencing the positions of the elements. It is possible to cite the context of the automobile, for example, with the assembly of engine components, or the insertion of electrical connectors in the electrical and electronic industry. It is known that a rigid hold of the two elements that are to be assembled does not make possible the success of a robotized assembly. It is often necessary to use at least limited compliance in directions that are perpendicular to the axis of insertion.

Furthermore, in the context of the collaborative handling, the safety of the human operator is of paramount importance. To ensure his safety, it is necessary not only to monitor the speed of the robotic system but also the motive forces that it exerts.

The existing technologies of the type called “artificial muscle” make it possible to respond at least in part to the problems and needs expressed above.

However, they are very difficult to implement and control, require difficult and tedious assemblies, and absolutely must be combined with additional kinematic guide means.

Finally, there are also industrial contexts where the environmental constraints are stringent and exclude the use of electromechanical technologies. These include, for example, the case of explosive environments, because of the handling of inherently dangerous products (explosive materials), or under specific conditions, such as, for example, the handling of powders as used in fields such as additive manufacturing. In this case, the use of technologies eliminating any active element on an electrical plane is a certain advantage.

This invention has as its object to propose a solution that makes it possible to overcome at least some of the drawbacks and the primary above-mentioned limitations.

For this purpose, it has as its object a pneumatic device for holding and moving an elongated object, with said device comprising a hollow body of general annular or tubular shape defining an axial direction, traversed by the elongated object at an axial passage and consisting of two end parts or segments arranged in a manner that is aligned in the axial direction and that forms jaws and a median or central part or segment that connects the two parts or segments that form jaws with one another, with relative ease of movement of one in relation to the other in the axial direction under the action of a controlled deformation of at least one portion of said median or central part or segment,

device characterized in that the various parts or segments of the hollow body define respective chambers,

in that the end part(s) or segment(s) that form jaws comprise elastically deformable membranes as wall portions that are designed to come into contact with the elongated object in the clamping phase,

and in that the chambers of the two end parts or segments are in fluid communication with the chamber of the median or central part or segment, each via at least one respective calibrated flow means, with the injection of pressurized gaseous fluid being carried out by means of a single feed line that is connected to a controlled source of pressurized fluid and that empties into the chamber of one of the two end parts or segments, based on the direction of translational movement that is desired for the elongated object that is present in the hollow body.

The invention will be better understood owing to the description below, which relates to preferred embodiments, provided by way of non-limiting example and explained with reference to the accompanying diagrammatic drawings, in which:

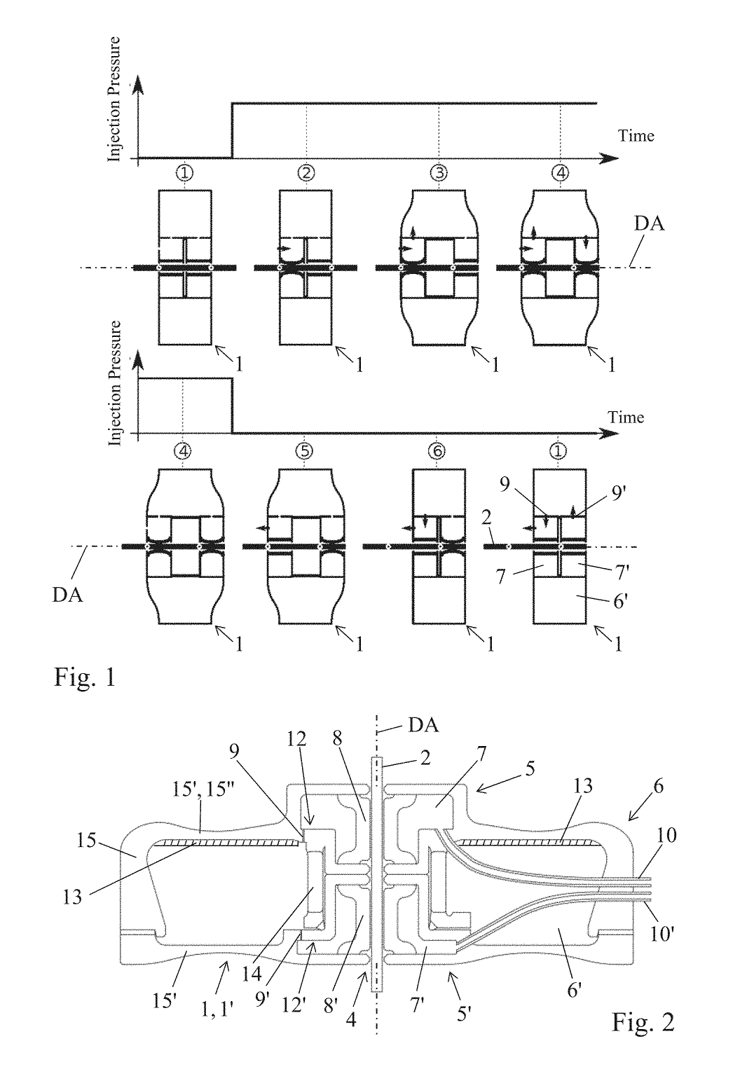

FIG. 1 illustrates, in the form of a series of diagrammatic representations and time charts, the successive stages of the kinematics developed by a holding and moving device according to the invention for carrying out an elementary movement (a sequential step) of an elongated object, in relation to the changes in the pressure of the gaseous fluid that is injected via a single feed line (50/50 cyclic injection/evacuation ratio);

FIG. 2 is a cutaway view of a first practical embodiment of a device according to the invention, having a flattened structure in the axial direction, of disk-like shape;

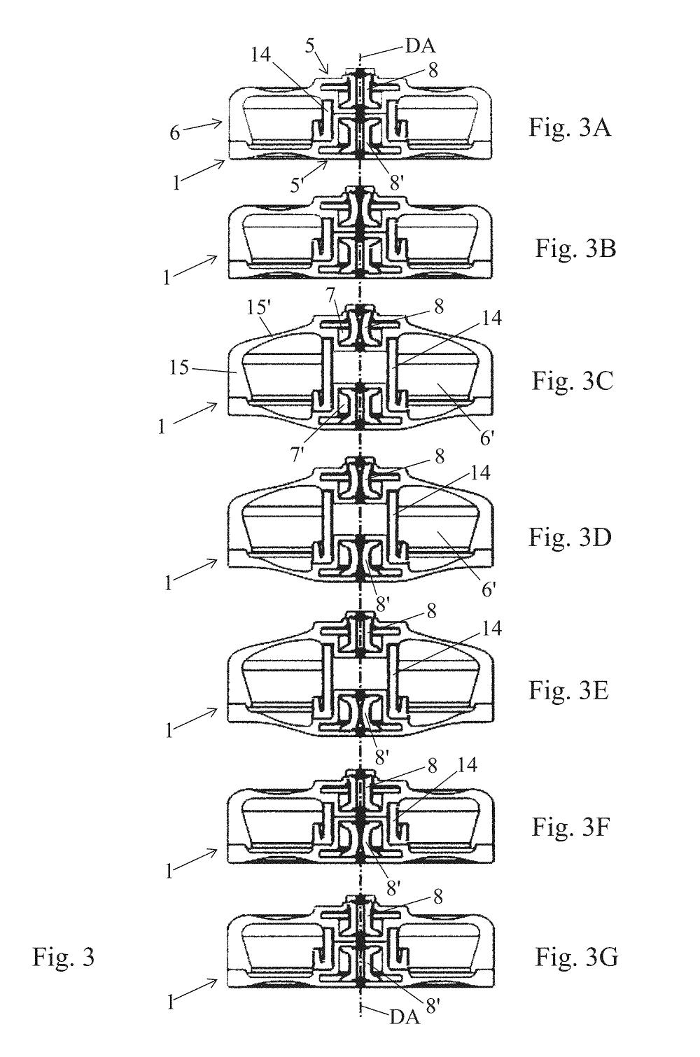

FIGS. 3A to 3G are cutaway views that are similar to that of FIG. 2, illustrating the various stages for modification of the device of FIG. 2 during the execution of the kinematic stages of an elementary movement (the moved object not shown);

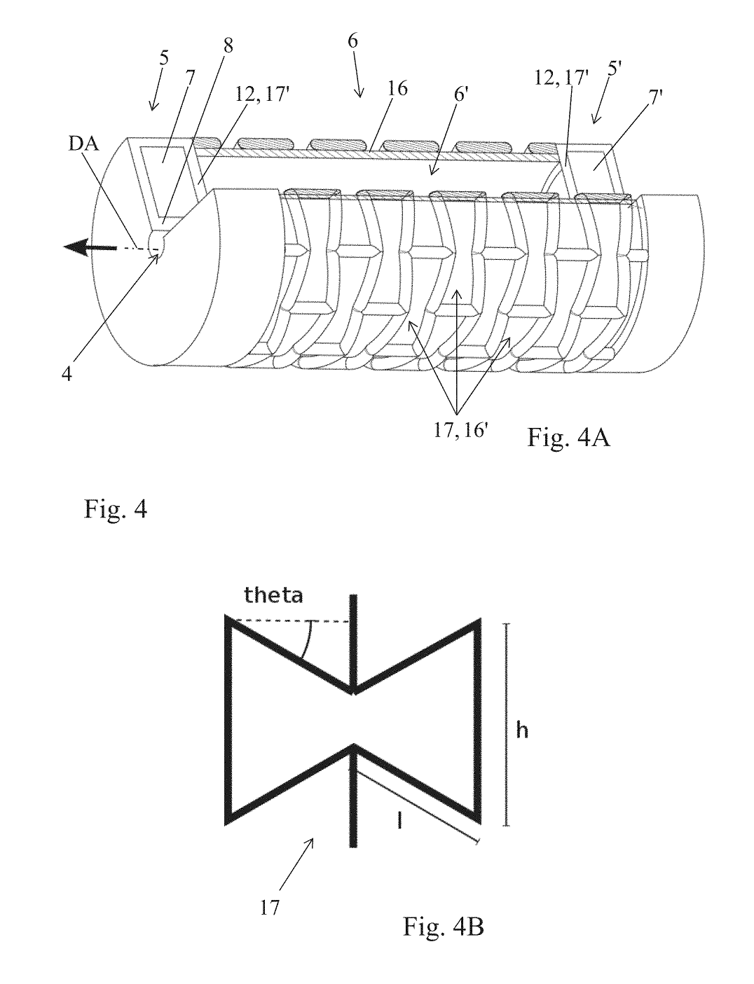

FIG. 4A is a diagrammatic perspective and partial cutaway view along an axial plane of a second embodiment of the invention in which the median part or segment comprises a material with an auxetic property;

FIG. 4B is a detail view, on a different scale, of an elementary formation of the pattern that forms the auxetic structure of the median part that is shown in FIG. 4A;

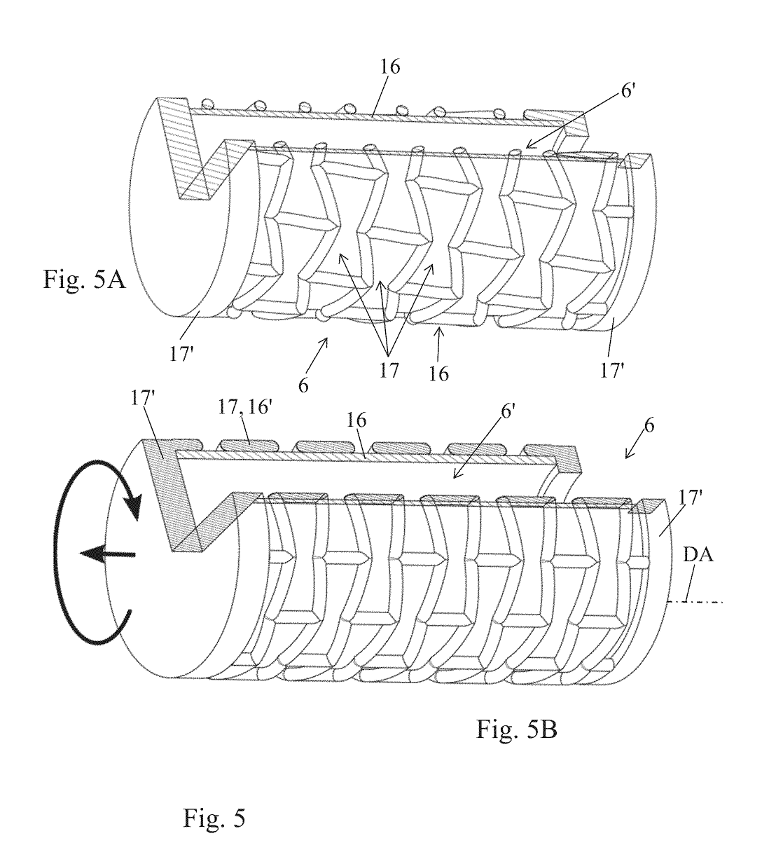

FIGS. 5A and 5B are diagrammatic perspective views of the median part of another variant of the second embodiment of the device according to the invention, integrating an auxetic material with a twisted structure and shown respectively in the absence of pressurized fluid injection (at rest: FIG. 5A) and after an operational internal pressure is reached (FIG. 5B);

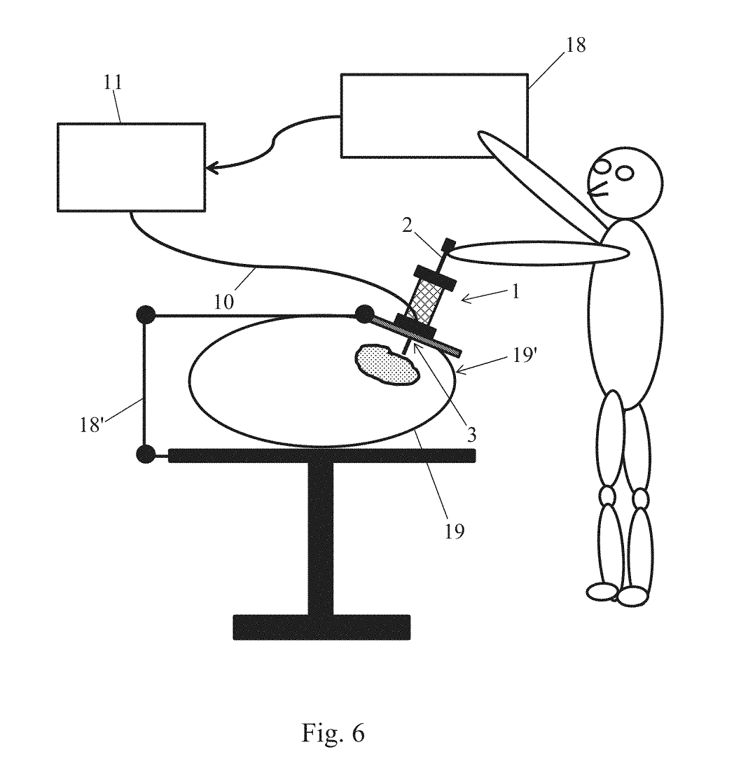

FIG. 6 is a diagrammatic representation that illustrates an example for implementing the device according to the invention within a medical context;

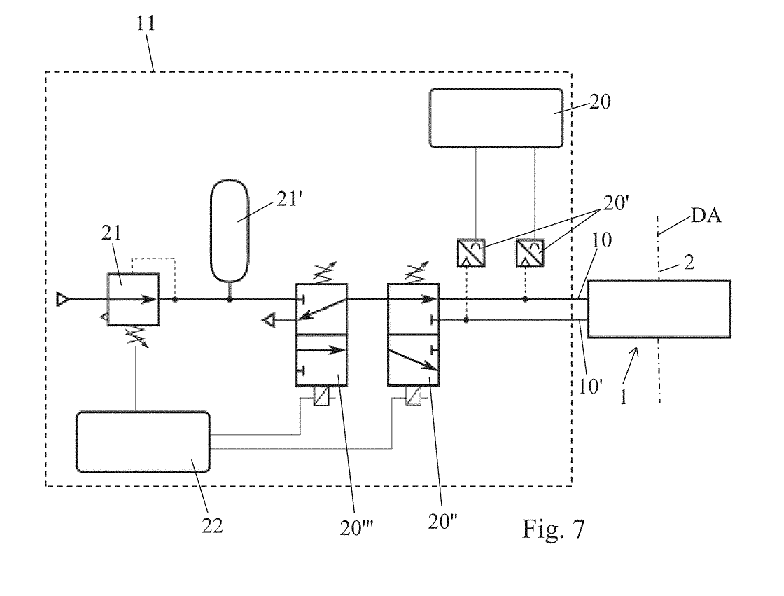

FIG. 7 is a block diagram of a controlled feed system for a device according to the invention, which can be part of a medical system shown in FIG. 6;

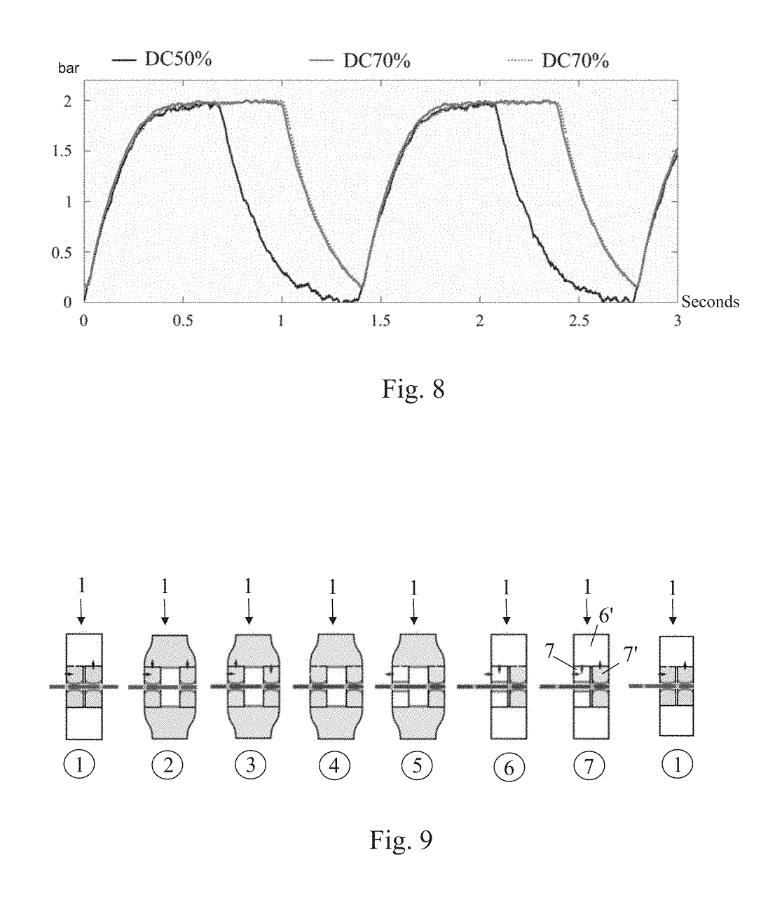

FIG. 8 shows pressure curves (in bar) based on time (in seconds) illustrating two air feed profiles of a device according to the invention with 50/50 and 70/30 cyclic injection/evacuation ratios;

FIG. 9 illustrates, in a manner similar to FIG. 1, the successive stages of the kinematics developed by a device according to the invention that is fed with a 70/30 cyclic ratio;

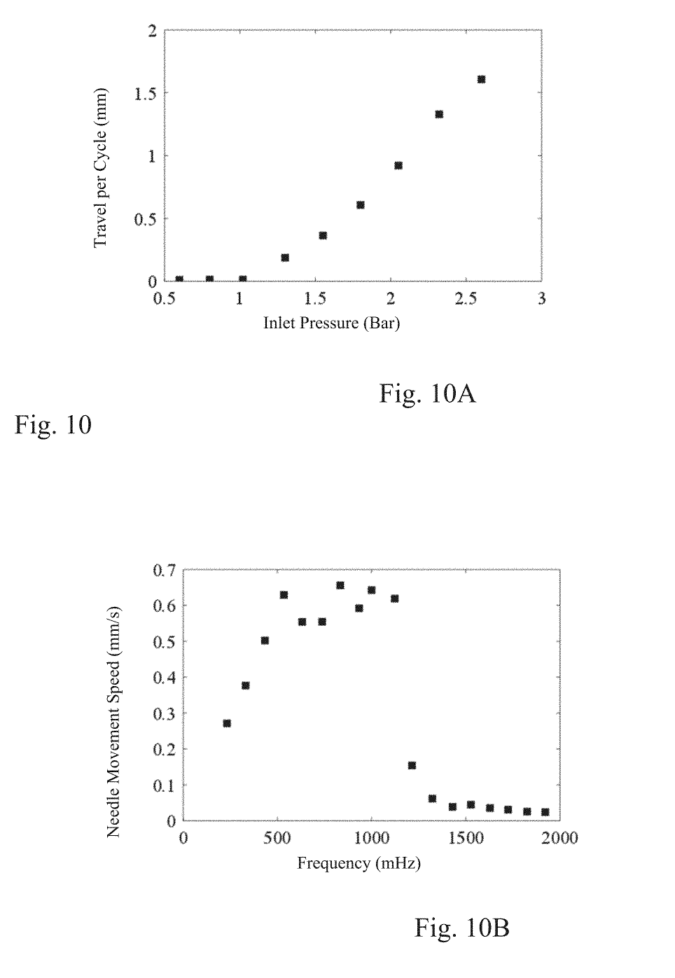

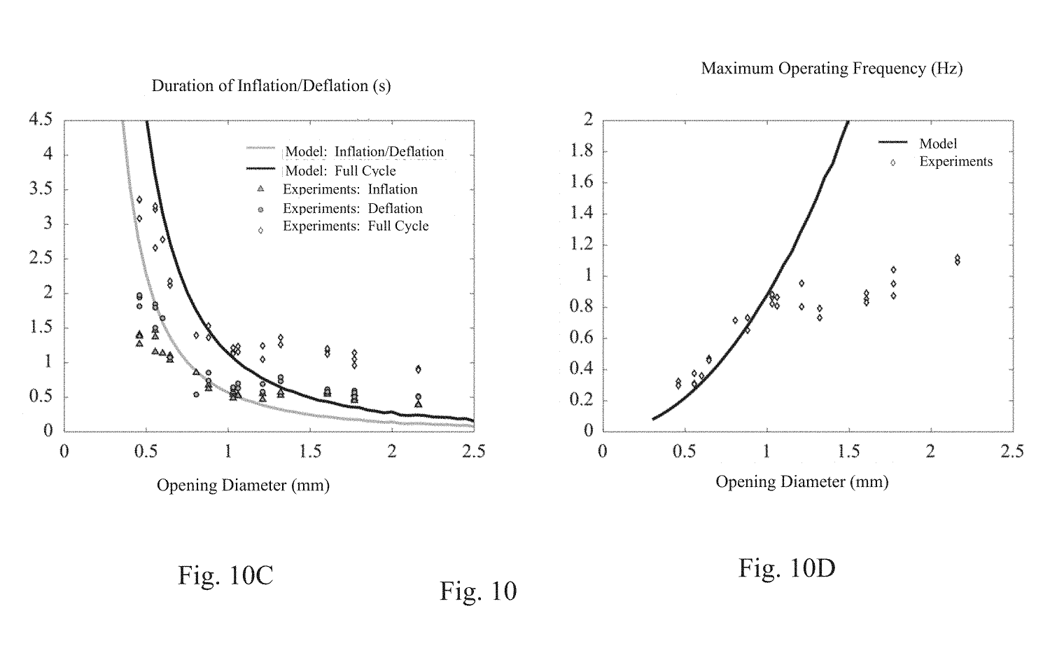

FIGS. 10A to 10D are curves that illustrate respectively: the travel per cycle based on the feed pressure (FIG. 10A); the speed of movement of the object being handled, based on the frequency of the feed cycles (FIG. 10B); the periods of inflation and deflation based on the diameter of the openings (FIG. 10C), and the maximum frequency of cycles based on the diameters of the openings (FIG. 10D), for a device as shown in FIGS. 2 and 3;

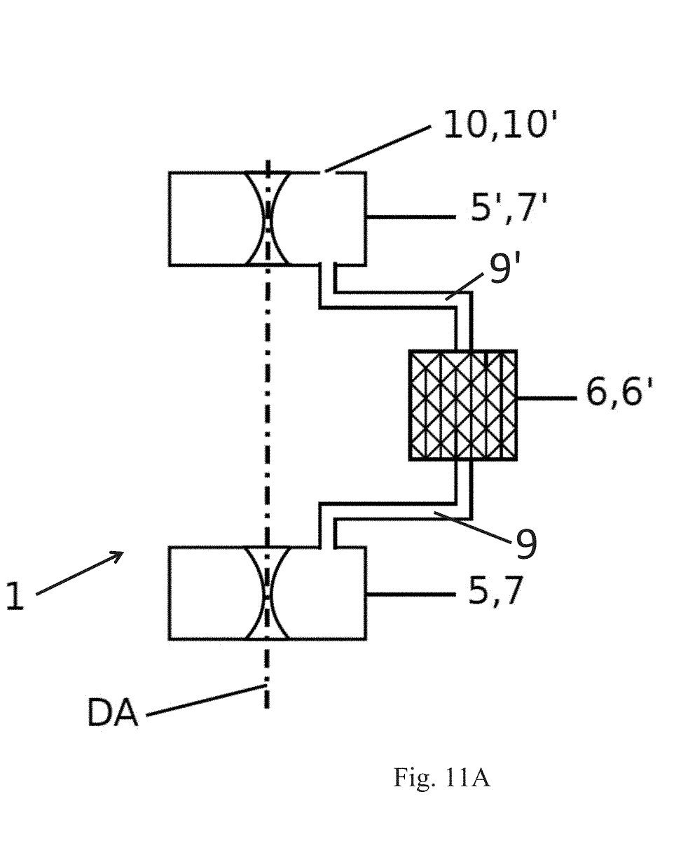

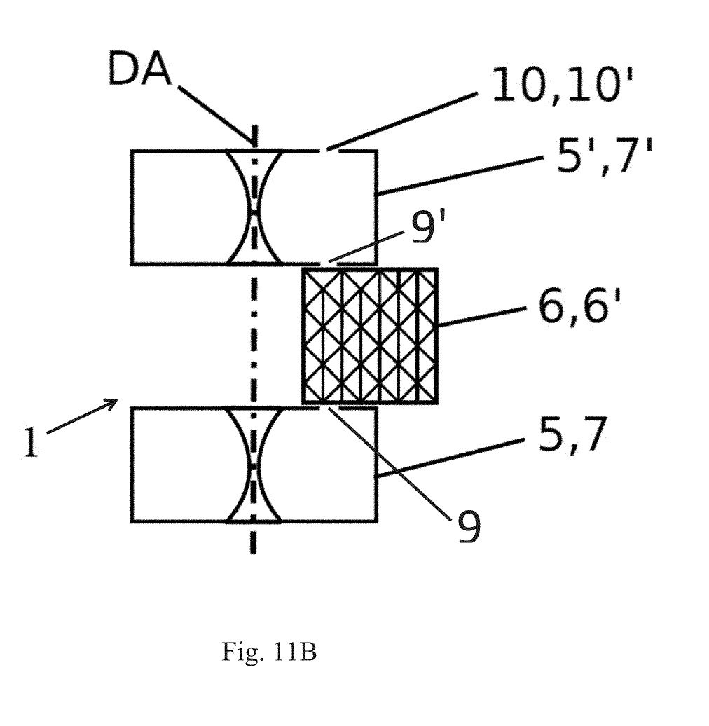

FIGS. 11A and 11B are diagrammatic and block representations of a device according to two variant embodiments of the invention, in which the median or central chamber or part is offset radially or laterally in relation to the end parts or terminal chambers that form jaws;

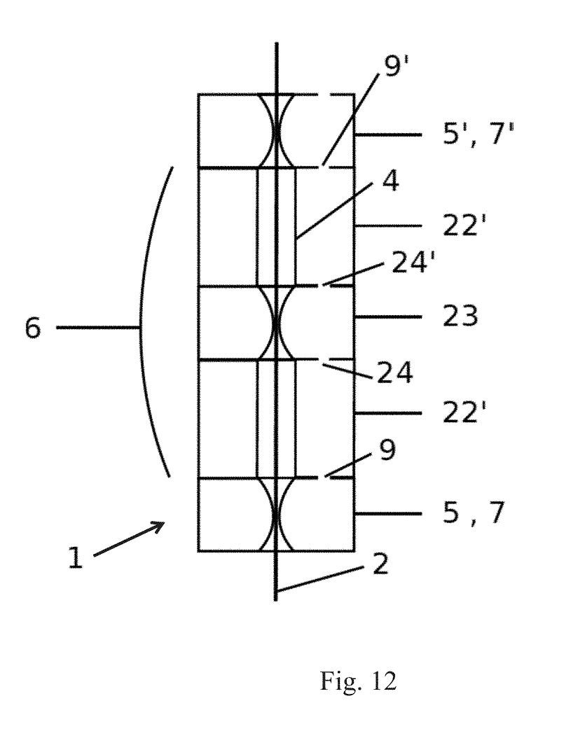

FIG. 12 is a diagrammatic cutaway representation and elevation representation of a device according to another embodiment of the invention;

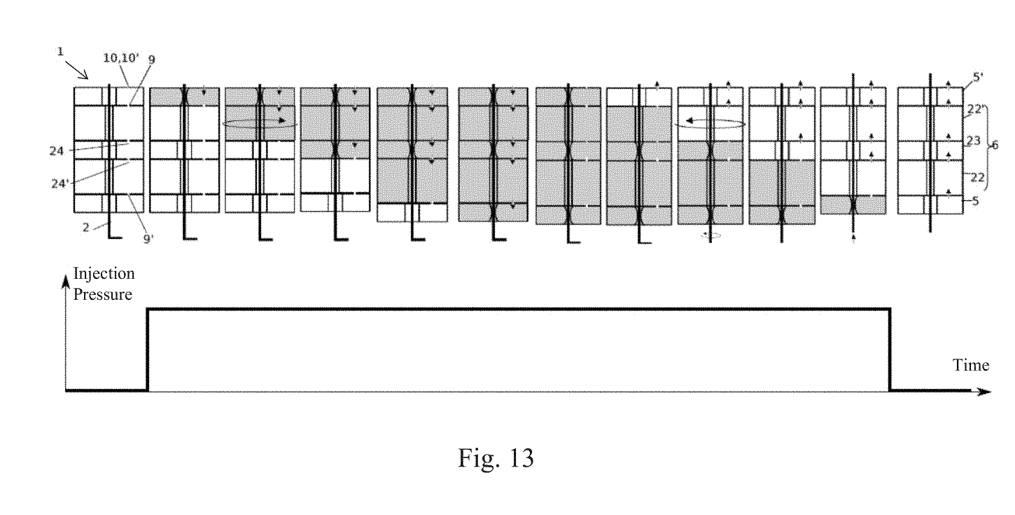

FIG. 13 illustrates, in the form of a series of diagrammatic representations and time charts, the successive stages of the kinematics developed by a device as shown in FIG. 12 (rotational movement and translational movement, successive sequences) with regard to the changes in the pressure of the injected gaseous fluid;

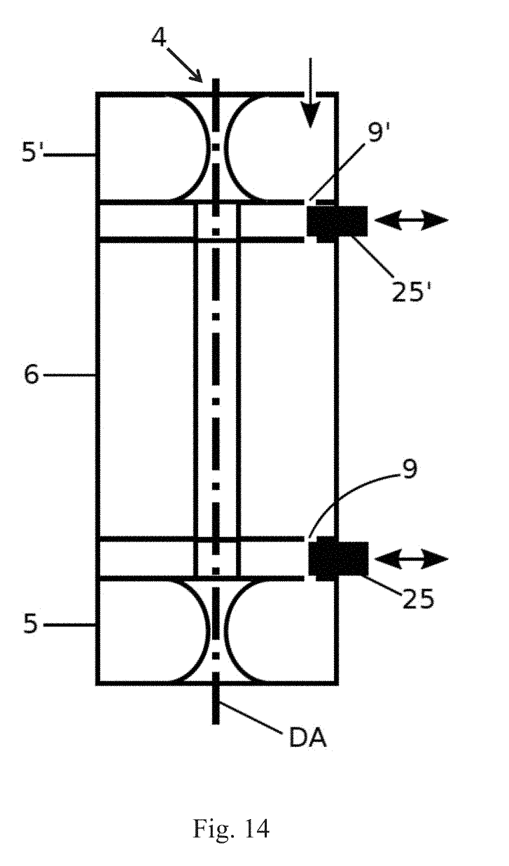

FIG. 14 is a diagrammatic cutaway view and elevation view, similar to those of FIGS. 1 and 12, of a device that is equipped with adjustment means of the calibrated flow means (openings that traverse the walls that are common to chambers that form jaws and to the median or central chamber);

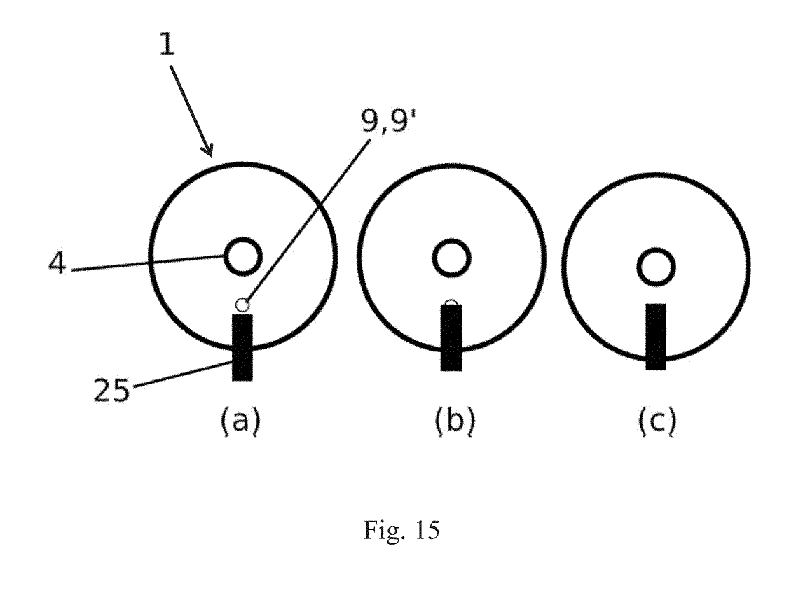

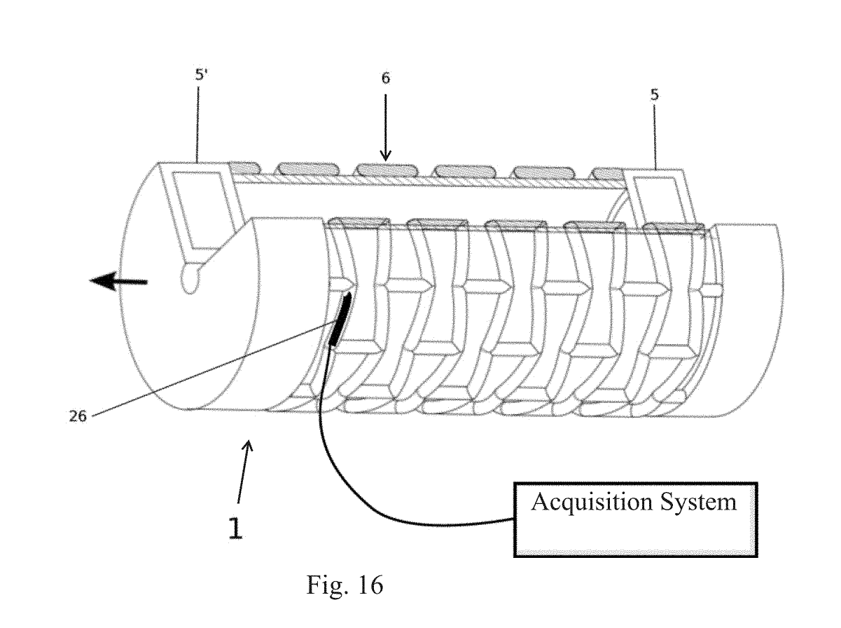

FIG. 15 is a top view of a wall that is common with a flow opening as shown in FIG. 14, with the adjustment means being shown in three different positions (a, b, c);

FIG. 16 is a view that is similar to that of FIG. 5, with the device being equipped with a sensor means;

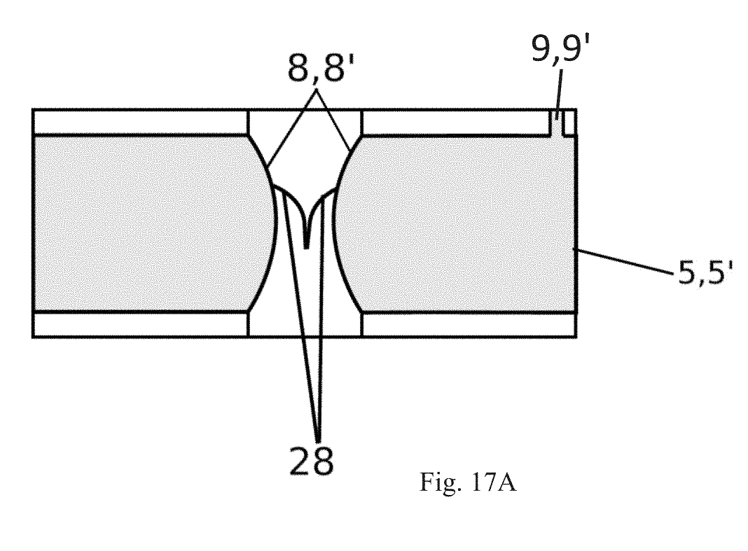

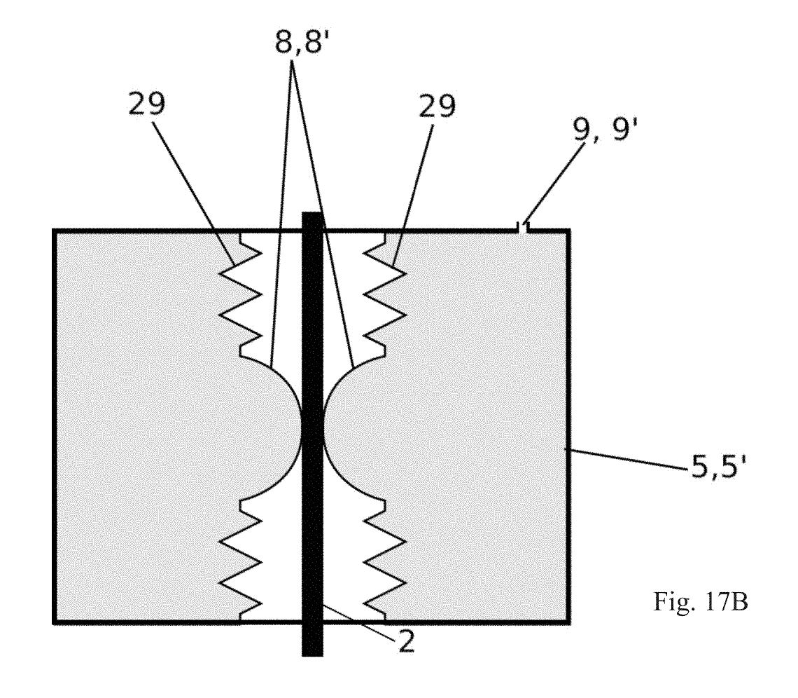

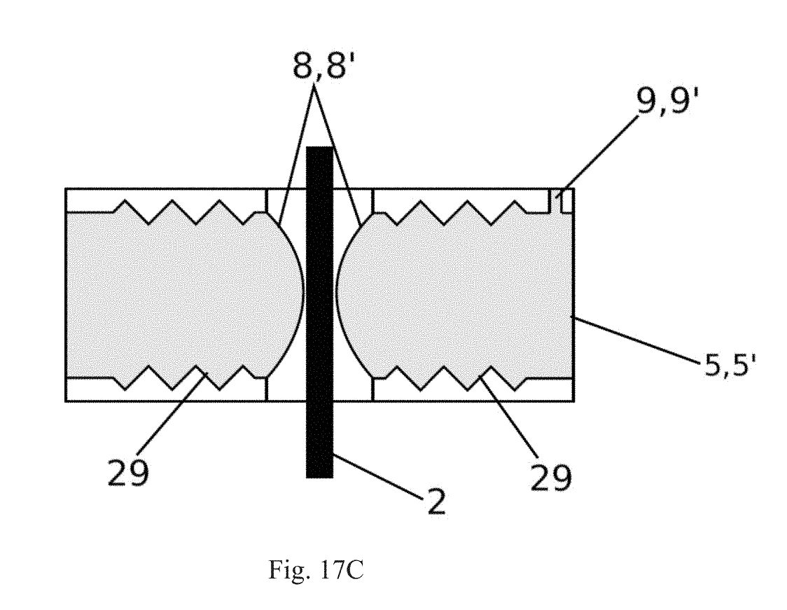

FIGS. 17A to 17C are diagrammatic cutaway views and elevation views of a terminal chamber or end part that forms jaws of a device according to the invention, illustrating various particular designs of the wall or of the internal deformable membrane of said part (FIG. 17A: unclamped state—FIGS. 17B and 17C: clamped state), and

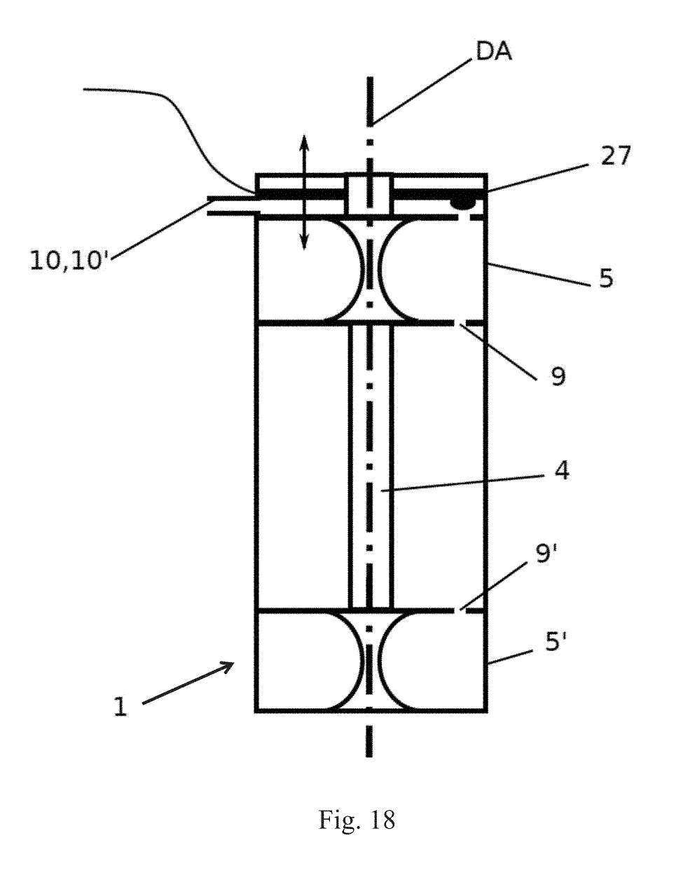

FIG. 18 is a diagrammatic cutaway view and elevation view of a device according to the invention as shown in FIGS. 1, 12 and 13, equipped with a solenoid valve for control of the injection of gaseous fluid.

FIGS. 1 to 4, 6, 9, 11 to 14, 16 and 18 all illustrate a pneumatic device 1 for holding and for automatic movement of an elongated object 2.

This device 1 comprises a hollow body 1′ of general annular or tubular shape that defines an axial direction DA, traversed by the elongated object 2 at the level of an axial passage 4, and consists of two end parts or segments 5 and 5′ that are arranged in an aligned manner in the axial direction DA and that form jaws and a median or central part or segment 6 connecting the two parts or segments that form jaws 5 and 5′ with one another, with relative ease of movement of one with regard to the other in the axial direction DA under the action of a controlled deformation of at least one portion 14, 15′ of said median or central part or segment 6.

In accordance with the invention, it is provided:

that the various parts or segments 5, 5′, 6 of the hollow body 1′ define respective chambers 7, 7′, 6′,

that the end part(s) or segment(s) 5, 5′ that form jaws comprise elastically deformable membranes as wall portions 8, 8′ designed to come into contact with the elongated object 2 in the clamping phase,

and that the chambers 7, 7′ of the two end parts or segments 5, 5′ are in fluid communication with the chamber 6′ of the median or central part or segment 6, each via at least one respective calibrated flow means 9, 9′, with the injection of pressurized gaseous fluid being performed by means of a single feed line 10, 10′ connected to a controlled source of pressurized fluid 11 and emptying into the chamber 7, 7′ of one 5 or 5′ of the two end parts or segments 5, 5′, based on the direction of translational movement that is desired for the elongated object 2 that is present in the hollow body 1′.

As FIGS. 1 to 4, 6, 9, 12 to 14, 16 and 18 show and consistent with a first design of the device 1, the end parts or segments 5, 5′ and the median or central part or segment 6 are aligned with one another, and the elongated object 2 traverses all of them, with the axial direction DA of the hollow body 1′ and the axis of translation of the elongated object 2 being combined.

The end parts or segments 5 and 5′ then have a concentric arrangement seen in the direction DA.

Consistent with a design variant of the invention, illustrated diagrammatically in FIGS. 11A and 11B, the end parts or segments 5, 5′ are aligned with one another, and the elongated object 2 traverses both of them, with the axis of translation of the elongated object 2 being combined with the axis of alignment of said end parts or segments 5, 5′ corresponding to the axial direction DA, with the median or central part or segment 6 being offset laterally or radially in relation to this direction DA, and the elongated object 2 not traversing it.

The median or central chamber 6′ may or may not share common walls with the terminal chambers 7 and 7′.

The gaseous fluid is preferably air under low pressure (preferably 1 to 5 bar), optionally treated, easily available in health establishments or industrial sites.

The elongated object 2 can consist of, for example:
Either a surgical instrument or tool of oblong shape and designed to pass through an opening or a tubular passage,
Or a piece, a tool or an instrument that is designed to be inserted into a recessed opening or that traverses a second or other piece.

For its handling by the device 1, the object 2 is to comprise at least one elongated part of cylindrical shape if it does not have a cylindrical elongated design overall.

Thus, the invention provides a device 1 that forms a linear actuator for any type of elongated piece, instrument or tool 2, with the ability (owing to the surface contact of the membranes 8, 8′) to grasp pieces, tools or instruments of various sizes and diameters firmly, without the latter having to undergo any modification (for achieving possible cooperation, a hypothetical coupling or interlocking) and without their outside surface being modified or degraded by their use with regard to the device 1.

The axial through passage 4 is advantageously sized based on larger tools or instruments 2 provided for handling by the device 1 (objects or pieces of larger diameters).

With the movement not being carried out with a continuous motion but in a sequential manner, the risks of uncontrolled movement and even of undesirable movement are limited (in frequency and in scale), and even eliminated. The safety of the motion, within a medical context, is therefore greatly increased. In addition, taking into account forces in play and the speed of execution of the successive elementary motions, a dominant and corrective human intervention is always possible.

In addition, taking into account the enveloping structure of the hollow body, or at least of the two end jaws 5 and 5′, around the oblong object 2 that is to be moved, holding the latter in a tube with a specified diameter is always ensured, while allowing freedom of movement framed in this cylindrical interior volume (axial passage 4). Actually, in the deflated state or at rest, the device 1, with its tubular passage 4 in which the tool or instrument 2 is placed, prevents too significant a displacement in relation to the axial direction DA, but allows a sufficient motion that makes it possible, for example, for a surgical needle that is planted in an organ to follow the physiological motion of the latter (rotational motion of the needle in relation to its point of insertion in the body of the subject 19) or else to be manipulated by the practitioner.

Furthermore, and consistent with a significant advantage provided by the invention, the feeding by a single line 10, 10′ (by direction of movement) greatly simplifies the connector, reduces the space requirement at the site for implementing the device 1, and facilitates the management of the fluid. Actually, the various consecutive kinematic stages that are necessary for achieving the movement of the object 2 by successive elementary steps are carried out naturally and without active intervention other than the injection and the stopping of the injection of pressurized fluid, because of the particular calibrated communication of the three chambers 7, 6′ and 7′ that determines the order, the duration and the intensity of the activation sequences (pressurization) and deactivation (depressurization) of the latter generating an “inch-worm”-type movement.

Finally, taking into account its pneumatic and structurally deformable nature, the device 1 in all of the circumstances of implementation (even inflated) has an at least minimum axial and/or radial compliance.

Advantageously, the end parts or segments 5, 5′ that form pneumatic jaws, respectively front and rear based on the direction of positive movement of the elongated object 2, delimit chambers 7, 7′ that are annular or rotationally toroidal in shape around the axial direction 41 DA and that have a structure that is essentially non-deformable—axially and radially—within the range of operational pressures of fluid that is injected during normal operation of the device 1, with the exception of elastically deformable membranes that form internal sleeves 8, 8′ that constitute through housings of the jaws for the elongated object 2, forming by alignment the axial passage 4 of the multi-chamber hollow body 1′.

Thus, the device 1 comprises two annular or toroidal chambers with an elastically deformable wall 8 or 8′, under internal pressure, radially toward the inside (pneumatic jaws function) and a median annular or tubular connecting chamber 6′, elastically deformable under internal pressure by axial elongation or extension of the hollow median segment 6 that delimits it.

Preferably, the median or central part or segment 6 has at least one common wall portion 12, 12′ with respectively each of the two end parts or segments 5, 5′, with each of said common wall portions 12, 12′ thus participating simultaneously in the partial delimitation of one of the terminal chambers 7, 7′ and with that of the median or central chamber 6′. In this case, the calibrated flow means 9, 9′ can consist of through holes that are made in said common wall portions 12, 12′ and that have specified shapes and sizes, based on the desired sequencing for the inflation and the deflation of the chambers 7, 6′, 7′ determining the amplitude and/or the speed of the translational movement of the elongated object 2 in question.

Consistent with a characteristic of the invention, the calibration of each of the respective flow means 9, 9′ between each of the terminal chambers 5, 5′ and the median or central chamber 6, which is optionally different and suitable for each means 9, 9′, is adjusted in such a way that, by passive monitoring of the circulation of the gaseous fluid that is injected via the single feed line 10, 10′, the two terminal chambers 5 and 5′ can be under an operational pressure that brings about an effective double clamping of the object 2 that is to be moved, when the median or central chamber 6 is under operational pressure and in its deformed state.

One skilled in the art understands that the passive monitoring of the flow of gaseous fluid between the various communicating chambers 6′, 7, 7′—both during the injection phases (inflation) and during the evacuation phases (deflation)—primarily depends on the sizing of the means 9 and 9′, namely the diameter of each of the holes 9 and 9′ within the framework of the preferred variant embodiment of the invention. These monitored flows that are determined by sizing at manufacture define the durations of inflation and deflation sequences of the chambers 6′, 7, 7′ during the two phases, with regard to, of course, the volumes of said chambers and their coefficient of expansion by deformation, with the pressures/negative pressures applied, with the nature of the gaseous fluid that is used (normally air), and with the mutual cyclic ratio of the inflation and deflation phases.

Another parameter of importance is the clamping force that is desired at each jaw 5, 5′.

For a device 1 as shown in FIG. 2, with membranes 8, 8′ having axial lengths of approximately 10 mm each and chambers 6, 7, 7′ having volumes of 10⁻³ to 10⁻⁴ m³ and fed with air under a pressure of approximately 2 bar, each of the two holes 9, 9′ advantageously has a diameter of approximately 1 mm, making it possible to produce a clamping force of approximately 8 to 10 N with a cyclic frequency of approximately 1 Hz and a cyclic pitch of approximately 1 mm, according to the experiments and tests carried out by the inventors.

A simulation based on the laws and rules of fluid mechanics and the elastic deformation of the bodies, easily within the scope of one skilled in the art, may make it possible to size the device 1 and its operating parameters based on the desired operational result.

The chronologically successive stages of a kinematic cycle of the device 1 as it is shown in FIGS. 2 and 11 by examples, producing a movement of an elementary step of the object 2, are illustrated in FIG. 1 (in gray: operational pressurized chambers or chambers at least under a deforming pressure):
Stage 1 (in duplicate in FIG. 1: beginning and end of the cycle): The device 1 is at rest and the hollow body 1′ is not subjected to any gaseous pressure (deflated state—at atmospheric pressure):
   The object 2 is not held rigidly and is not integral with the hollow body 1′; it can be manipulated by the user directly (limited displacement).
Stage 2: Beginning of a cycle of translational movement: compressed air is injected via the feed line 10 into the chamber 7 of the front-end part 5, and the tubular membrane 8 of these jaws is applied internally (with a predetermined pressure) against the local surface opposite to the object 2 (grasped by the latter).
Stage 3: When the first jaw 5 is essentially inflated at the operational pressure thereof (therefore after flow of a certain duration), the air escapes via at least one calibrated opening 9 into the chamber 6′ of the median part 6, which is extended in the axial direction DA under the air pressure and separates the two jaws 5 and 5′.
Stage 4 (illustrated in duplicate in FIG. 1): When the median chamber 6′ is inflated at its operational pressure, i.e., when the median part 6 has reached its degree of nominal elongation, the air escapes via at least one calibrated opening 9′ toward the chamber 7′ of the rearward end part 5′, and the tubular membrane 8′ of these jaws is closely applied (with a predetermined pressure) against the local surface of the object 2 opposite (local surface contact) when the operational pressure is reached in the chamber 7′ (dual grasping of the object: holding by the two jaws 5 and 5′ with a spacing that is defined by the elongation of the median part 6).
Stage 5: The supply of compressed air is stopped, and the pressurized air is evacuated through the feed line 10 that acts as an evacuation line. The chamber 7 of the front-end part 5 empties, and the corresponding jaw releases its grip on the object 2.
Stage 6: The pressurized air that is contained in the chamber 6′ of the median part 6 is evacuated, and this part 6 regains its shape at rest (not elongated). The two jaws 5 and 5′ move toward one another, and the rear jaw 5′, still engaged with the object 2, moves it through the front jaw 5 (kept stationary).
Stage 7: Finally, the chamber 7′ of the rearward end part 5′ also empties, and the corresponding jaw releases the object 2, which is free in the tubular axial passage 4 (this is a situation that is identical to that of Stage 1).

The above-mentioned cycle described with regard to the representations of FIG. 1 is based on a cyclic ratio of value 1 between the fluid injection and evacuation phases (for each cycle corresponding to an elementary movement of the object 2, the time of each of the two phases represents 50% of the duration of the cycle) and comprises a transitory stage for passage between two consecutive cycles during which the object 2 is released (Stages 1 and 7 of FIG. 1).

When, for example in the case of a depression with elastic return or resistance to the depression, the goal is to maintain a continuous grip during the entire depression motion, the cyclic ratio between the two phases can be modified (for example, 70/30) in such a way that the inflation of the front jaws 5 begins before the deflation of the rear jaws 5′ is achieved, and even before it has begun. In this case, the device 1 is engaged permanently with the object 2 during the entire movement by cyclic pitch of the latter.

So as to amplify the elongation phenomenon of the median or central part 6 and consequently the movement step per kinematic cycle, it may be provided that this part 6 is at least partially formed by or covered with a layer 13 of an auxetic material, i.e., with a negative Poisson's ratio. The auxetic nature of said material can originate from the structure of the latter and/or the very nature of said material.

In accordance with a preferred practical variant embodiment, the multi-chamber hollow body 1′ with its three outputs or segments 5, 5′ is entirely made of polymer material(s), preferably both as a one-part piece, or made integrally, for example by three-dimensional printing, with the deformable membranes 8, 8′ of the jaws 5, 5′ optionally being connected by over-molding of a specific distinct polymer material.

Thus, the device 1 can be used in any context of medical imaging and can be manufactured for a low cost, optionally in the form of a disposable product.

In an advantageous manner, nevertheless, the polymer materials that are used, for example rigid acrylate resin (of an elasto-plastic nature) for the rigid walls of the hollow body 1′ and flexible acrylate resin (of an elastomeric nature) for the elastically deformable walls of the latter, are of a nature to withstand sterilization and are compatible with use in a sterile chamber.

As a variant, the rigid material can be a polypropylene or a polyoxymethylene, and the flexible material can be a silicone.

The walls of the hollow body 1′ can also be produced from an alloy with shape memory, optionally with a specific configuration of said walls, for a deformation that is controlled and reversible based on the internal pressure in the chambers 6′, 7, 7′. An elastically flexible material can be connected by over-molding to form the membranes 8, 8′.

For the purpose of using a device 1 that can move the elongated object 2 both ways in the axial direction DA, without modifying the mutual arrangement thereof, and therefore being able to concatenate insertion and extraction motions, it can be provided that a conduit 10, 10′ that is able and is designed to form selectively a feed line or a drain line is connected to each of the chambers 7, 7′ of the end parts or segments 5, 5′ (FIG. 2).

According to a first embodiment of the invention, illustrated in FIGS. 2 and 3, the median or central part or segment (6) defines an annular or toroidal chamber 6′ and extends peripherally and externally radially around the two end parts or segments 5, 5′ to produce a multi-chamber hollow body 1′ of disk-like shape. This part or this segment 6 connects these parts or segments 5, 5′ to one another by an elastically deformable bond in the axial direction DA, with the two end parts or segments 5, 5′ being in mutual contact in said axial direction DA in the deflated state of the median or central part or segment 6 and the two end parts or segments 5, 5′ being located at a distance from one another in the inflated state of said median or central part or segment 6, under a pressure that is sufficient to bring about a deformation of the latter. The deformation of the latter bringing about a nominal spacing of inflatable jaws 5 and 5′ is achieved when the operational pressure value is reached in the median or central chamber 6′.

In this embodiment, the device 1 at rest has a longitudinal extension in the direction DA of minimum value, for a contact surface of the jaws 5, 5′ with the object 2 that is maximum.

Actually, the median part 6 and its associated chamber 6′ extend around jaws 5 and 5′ and have an axial dimension that is less than the total of the axial dimensions of the two jaws 5 and 5′. Thus, at rest, the axial dimension of the device 1 corresponds to the sum of the axial dimensions of the two jaws 5 and 5′, which are in contact axially.

In accordance with an advantageous practical design with regard to the first above-mentioned embodiment, the median or central part or segment 6 of the multi-chamber hollow body 1′ consists of, on the one hand, a portion of internal tubular wall 14 that can be deformed elastically in the axial direction DA and that connects the two end parts or segments 5, 5′ between them, and, on the other hand, an outside wall portion with a U-shaped cross-section 15, for rotation around the axial direction DA and that forms—by additionally cooperating with the portion of internal tubular wall 14—the annular or toroidal median or central chamber 6′ of the hollow body 1′, with the two wings 15′ of the U also being connected by their free ends to the two end parts or segments 5, 5′ and being elastically deformable by bending toward the outside of the U in the axial direction DA, under the action of an adequate overpressure in the central or median chamber 6′.

The inflation of the median or central chamber 6′ consequently brings about an elastic spacing of the wings 15′ of the rotational structure with a U-shaped cross-section 15 and simultaneously an elastic stretching of the internal tubular wall 14 in the axial direction DA, producing a spacing of the two end parts or segments 5 and 5′ between them.

Based on the desired kinematics, one of the two wings 15′ (for example, the front wing 15′ that is associated with the end part 5) can have greater deformability and can even be the only deformable wing. A simultaneous elastic deformation of the front and rear wings 15′ makes it possible to increase the cyclic pitch of the device 1 (see FIGS. 3E and 3F).

The median or central part or segment 6, in addition to defining a central or median chamber 6′ that expands under pressure, produces a double elastic bond between the two end parts or segments 5 and 5′.

The openings 9 and 9′ for fluid communication are made in the wall portions 12 and 12′ that are located between the wings 15′ and the sites for making the tubular wall 14 integral with the end parts or segments 5 and 5′, at the rigid walls of the latter that form the annular chambers 7 and 7′ with the membranes 8 and 8′.

For the purpose of optionally producing a more significant movement in each cycle, the areas 15″ of the outside wall portion with a U-shaped cross-section 15 that correspond to the wings 15′ of said U are formed by or covered with, preferably on their internal face, a layer 13 made of auxetic material (FIG. 2) or with an auxetic property.

By way of practical example of the design of a device 1 that is produced by the inventors, on the basis of the embodiment that is shown in FIGS. 2 and 3, the following dimensions and coefficients can be applied:
Outside dimensions of the device 1:
   Outside diameter: 74 mm
   Outside thicknesses: 24 mm
Jaws 5 and 5′:
   Volume at rest: 2.1×10⁻⁶ m³
   Inside diameter at rest: 2.0×10⁻³ m
   Needle diameter: 1.8×10⁻³ m
   Length of membranes 8, 8′: 10.0×10⁻³ m
   Pressure before contact: 0.5×10⁵ Pa
Median chamber 6:
   Volume at rest: 4.2×10⁻⁵ m³
   Coefficient of volumetric rigidity: 1.3×10⁻¹¹ m³/Pa
   Coefficient of rigidity in motion: 4.5×10⁻⁹ m/Pa
Holes 9 and 9′:
   Diameter: 1.0×10⁻³ m
   Discharge coefficient: 0.577

By designing and by using a device 1 that is sized as indicated above and by modeling its behavior, the inventors have been able to establish the curves of FIGS. 10A to 10D, which indicate the typical values and limits and are optimum in terms of the sizing of the openings 9, 9′ for communication between chambers, injection pressure, cyclic frequency and cyclic pitch. Starting from these values, it is obvious for one skilled in the art to implement dimensional variants of the device 1.

According to a second embodiment of the invention, as shown in FIGS. 4 and 5, the central or median part or segment 6 can comprise a cylindrical tubular chamber 6′ that extends in the axial direction DA and the rotational direction around the latter, with said chamber 6′ comprising an elastically deformable outside wall 16 and being in calibrated fluid communication with the chambers 7, 7′ of the end parts or segments 5, 5′. Said wall 16 is covered on the outside by a cylindrical structure with an auxetic property 16′, which is formed by a gridded network with a repetitive pattern of elementary formations 17 and which rests on and is integral with two opposite disk-like stops 17′. These stops 17′ are also connected to the above-mentioned deformable wall 16 and form common wall portions 12, 12′ with end parts or segments 5, 5′ or are connected to the latter. The above-mentioned constituent components of the median part or segment 6 are mutually arranged in such a way that the operational pressurization of the median or central chamber 6′ brings about—by action of the wall 16 on the network 16′—a mutual spacing of the disk-like stops 17′ in the axial direction DA and therefore end parts or segments 5, 5′ that are respectively integral with said stops 17′.

In accordance with an advantageous practical design, the elementary formations 17 together constitute a network 16′ with an inverted honeycomb pattern and are arranged in several consecutive rows in the direction DA.

When a more complex motion than a simple translational movement is sought, it can be provided, for example, that at rest, the auxetic network 16′ is twisted around the axial direction DA, with an operational pressurization of said network bringing about a relative translational movement and a relative rotational movement between the two end parts or segments 5 and 5′ (FIGS. 5A and 5B, in contrast to the variant of FIGS. 4A and 4B whose network 16′ is not twisted).

FIG. 4B shows the essential dimensional parameters of the elementary formations 17 that define the geometry and the performances of deformations of the auxetic network 16′. By way of illustrative example and by selecting theta=10 degrees, 1=11 mm, and h=6.8 mm, a network 16′ that comprises bands of four consecutive elementary formations 17 on the circumference and ten such bands of repeated formations 17 on the axial length of the network 16′ makes it possible to carry out cyclic movements of approximately 3 mm under a pressure of approximately 1 bar.

Consistent with another design variant of the invention, illustrated in FIGS. 12 and 13 and allowing a sequential motion of the object 2, the central part or segment 6 can comprise three contiguous functional chambers or parts 22, 22′, 23, namely, on the one hand, two chambers 22, 22′ that are able and are designed to ensure a function of movement under the action of a deformation of at least one portion of their wall resulting from a modification of their internal pressure, and each coupled respectively to one of the end parts or segments 5, 5′ that form inflatable jaws, with which they are respectively in fluid communication by a calibrated flow means 9, 9′, and, on the other hand, a median functional part or chamber 23 that forms jaws, located between the two above-mentioned functional chambers or parts 22 and 22′ and in fluid communication by a respective calibrated flow means 24, 24′ with each of them, with all of the five parts 5, 5′, 22, 22′, 23 forming a cylindrical structure.

Within the framework of this design variant, it can be provided that a first of the two functional chambers 22 is configured to ensure a rotational movement around the axial direction DA of the elongated element 2 that is clamped by one 5 of the two end jaws 5, 5′ and that the second of the two functional chambers 22′ is configured to ensure a translational movement in the axial direction DA of the elongated object 2 that is clamped by the median jaw 23 and the other end jaw 5′.

The resulting sequenced motion is shown in FIG. 13 by way of example.

Such a double device 1 functionally and structurally corresponds to the agglomeration of two devices 1 that are coupled axially (one for the rotation and one for the translation), with the central jaw 23 being shared and common to both.

So as to be able to vary in particular the cycle durations and/or the speeds of certain optional phases, the device 1 can comprise, as FIGS. 14 and 15 show, at least one adjustment means 25 of at least one calibrated flow means 9, 9′, 24, 24′, in particular its passage section, with such an adjustment means 25 preferably being combined with each flow means 9, 9′, 24, 24′.

Advantageously, the or each adjustment means 25, with manual or motorized actuation, comprises a movable variable sealing element of the calibrated opening 9, 9′, 24, 24′ that forms the flow means in question.

The above-mentioned adjustment can be done at the beginning of the cycle and can be constant during the cycle or during two phases of the same cycle.

As shown diagrammatically in FIG. 16, the device 1 can also comprise a sensor means 26, connected to or integrated into the median or central part or segment 6, and is able and designed to deliver a signal based on the elongation of this part or this segment in the axial direction DA and/or around the latter.

This means 26 is connected to an acquisition system, and the information that is provided is, for example, used by the control system of the device 1 to monitor its operation.

As FIG. 17A shows, it may also be provided that the elastically deformable internal membranes 8, 8′, 23′ of the parts or segments 5, 5′, 23 that form jaws, defining a gripping tube 4 and coming into contact with the elongated object 2, comprise protruding formations 28 or surface texturing ensuring a flexible centered hold of the elongated object 2 in the deflated and retracted state of said jaws, such as, for example, lips.

Likewise, and as illustrated in FIGS. 17B and 17C, the invention can also provide that the wall of at least one of the parts or segments 5, 5′, 23 that form jaws, preferably the walls of all of these parts, comprise(s) elastically deformable portions 29 in the inflated, active or clamped state of said jaw(s), providing a radial and/or axial structural compliance that is at least limited to the elongated object 2 that is grasped by this (these) jaw(s).

These portions 29, for example in bellows form, thus allow increased radial or axial compliance.

So as to provide a complete integrated operating module, of small size, the device 1 can comprise a solenoid valve 27 for control of the injection of fluid, structurally coupled to one 5 of the end parts or segments that form jaws 5, 5′.

One skilled in the art understands that the device 1 according to the invention makes it possible to monitor the translational motion that is one of the basic motions of any robotic system, and at the same time produces the drive unit of this motion. The integration of two functions into one component, owing to the rigidity and the design of the device 1 in the directions other than the direction of motion, makes possible a unique compactness.

The proposed device 1 also inherently offers an axial compliance, tied to the characteristic of the central chamber 6′ that can be modified in design. This compliance is an important element for safety as disclosed above.

Additional design compliances can be integrated by particular configurations of the parts 5, 5′ and 6 as indicated above; the proposed embodiments based on polymer materials and on the excellent power-to-weight ratio of pneumatic technologies make it possible, for example, to propose a very light component for shaping low-weight robotic arms.

Finally, the device 1 forms an actuator that constitutes a safe and reliable means for monitoring motive forces by simply modulating a value that is the pressure inside the unit of the central chamber and jaws: this is in fact a particularly reliable mechanical force limiter.

The invention also relates, as FIG. 6 diagrammatically shows, to a system for controlled sequential holding and moving of an elongated object 2, such as a surgical or medical instrument or tool of oblong shape, designed to be inserted into the body of a patient 19.

This system comprises, on the one hand, at least one pneumatic holding and moving device carried by a specific support 18′ or made integral with the patient 19 and placed in direct contact with the skin 19′ of the latter, on the other hand, at least one pressurized gaseous fluid source, and, finally, a man-machine control interface 18, optionally being part of a medical installation.

This system is characterized in that said at least one pneumatic holding and moving device consists of a device 1 as described above, with its hollow body 1′ being held at its front-end part 5.

The invention also relates to a controlled, preferably automated, system for insertion and optionally assembly or mounting, by translational motion, optionally combined with a rotational motion of an elongated object 2, such as a structural piece, tool or instrument, in a passage, an opening or a receiving housing adapted for another object, piece, part, element or the like (not shown).

This system is characterized in that it comprises at least one pneumatic device 1 for guided holding and moving as described above, with said device 1 being mounted on an optionally movable support means, such as a robot arm or a collaborative robot, with said device 1 being an integral part, if necessary, of said robot arm or robot.

For the purpose of increasing the thrust force that can be applied to the object 2, it can be provided that the system comprises at least two pneumatic holding and moving devices 1, arranged in an aligned manner according to their axial direction DA (i.e., the axial directions DA of the devices 1 are combined) and each traversed by the elongated object 2 to hold and move automatically on command, with said devices 1 being fed with pressurized fluid in a coordinated manner (not shown).

By way of example, FIG. 7 illustrates a controlled feed system that forms a fluid source (here, the pressurized air) for the device 1. The system that is shown comprises: a measurement interface 20 that is connected to pressure sensors 20′ that monitor the lines 10, 10′; a valve 20″ for selecting the direction of movement; a valve 20′″ for generating the feed pressure profile over time; a pressure regulator 21 that is connected to the feed network and combined with a battery 21′; a device 22 for monitoring and guiding the system.

Finally, the object of the invention is also a method for inserting an elongated instrument or tool into the body of a subject by means of a system that is mentioned above.

This method is characterized in that it consists in installing at least one pneumatic holding and moving device 1 by aligning its axial direction DA with an inlet opening made in advance in the skin 19′ of the subject 19, then installing the instrument or the tool in the axial passage 4 of the hollow body 1′ of the device 1 up to a predetermined insertion position, then gradually and sequentially depressing said tool or instrument 2 by carrying out successively, for each elementary step of movement, a pressurized fluid injection phase and a phase for evacuating said fluid, with the injection phase being interrupted when the rear terminal chamber 7′ of the multi-chamber hollow body 1′ has reached an internal pressure that corresponds to its operational pressure, and repeating the cycle of the two above-mentioned phases as many times as necessary to reach the translational movement amplitude, and even the degree of depression, that is desired.

Finally, the invention also relates to a method for inserting an elongated object 2, such as a structural piece, tool or instrument, into an opening or a passage of a piece by means of the above-mentioned controlled system.

This method is characterized in that it consists in installing at least one pneumatic holding and moving device 1 by aligning its axial direction DA with the inlet opening of the opening or the passage in question, with the elongated object 2 being in place in the axial passage 4 of the hollow body 1′ of the device 1, then gradually and sequentially depressing said object 2 by successively carrying out, for each elementary step of movement, a pressurized fluid injection phase and a phase for evacuating said fluid, with the injection phase being interrupted when the rear terminal chamber 5′ of the multi-chamber hollow body 1′ has reached an internal pressure that corresponds to its operational pressure, and repeating the cycle of the two above-mentioned phases as many times as necessary to reach the amplitude of the translational movement, and even the degree of depression, that is desired.

When a continuous engagement of the device on the object 2 throughout the depression motion is desired, it may be provided that the injection phase of the next cycle begins while the evacuation phase of the cycle currently under way has yet to be completed (see FIGS. 8 and 9).

The difference between the kinematics of FIG. 1 (with release of the object 2) and that of FIG. 9 (without release) emerges from a comparison of representations of Stages 1 of FIG. 1 (release phases) with Stages 1 and 7 of FIG. 9. In FIG. 9, it is noted that when the chamber 7′ begins to empty (clamping still in effect), the chamber 7 is already in the process of inflating (Stage 7). The clamping at the jaws 5 will then be tightened while the clamping at the jaws 5′ continues to slacken to allow deployment by axial elongation of the chamber 6′ and the sliding of the jaws 5′ on the object 2 (Stage 2).

Of course, the invention is not limited to the embodiments described and shown in the accompanying drawings. Modifications can still be made, in particular from the standpoint of the composition of the various elements or by substitution of technical equivalents, without thereby exceeding the scope of protection of the invention.

1. A pneumatic device for holding and moving an elongated object, said device comprising a hollow body of general annular or tubular shape defining an axial direction, traversed by the elongated object at an axial passage and consisting of two end parts or segments arranged in a manner that is aligned in the axial direction and that forms jaws and a median or central part or segment that connects the two parts or segments that form jaws with one another, with relative ease of movement of one in relation to the other in the axial direction under the action of a controlled deformation of at least one portion of said median or central part or segment,
wherein,
the various parts or segments (5, 5′, 6) of the hollow body (1′) define respective chambers (7, 7′, 6′),
the end part(s) or segment(s) (5, 5′) that form jaws comprise elastically deformable membranes as wall portions (8, 8′) that are designed to come into contact with the elongated object (2) in the clamping phase, and
the chambers (7, 7′) of the two end parts or segments (5, 5′) are in fluid communication with the chamber (6′) of the median or central part or segment (6), each via at least one respective calibrated flow means (9, 9′), with the injection of pressurized gaseous fluid being carried out by means of a single feed line (10, 10′) that is connected to a controlled source of pressurized fluid (11) and emptying into the chamber (7, 7′) of one (5 or 5′) of the two end parts or segments (5, 5′), based on the direction of translational movement desired for the elongated object (2) that is present in the hollow body (1′).

2. The pneumatic device according to claim 1, wherein the end parts or segments (5, 5′) that form jaws, respectively front and rear based on the direction of positive movement of the elongated object (2), delimit chambers (7, 7′) that are annular or rotationally toroidal in shape around the axial direction (AD) and that have an essentially non-deformable—axially and radially—structure, within the range of operational pressures of fluid that is injected by normal operation of the device (1), with the exception of elastically deformable membranes that form internal sleeves (8, 8′) that constitute through housings of the jaws for the elongated object (2), which form by alignment the axial passage (4) of the multi-chamber hollow body (1′).

3. The pneumatic device according to claim 1, wherein the median or central part or segment (6) has at least one common wall portion (12, 12′) with respectively each of the two end parts or segments (5, 5′), with each of said common wall portions (12, 12′) thus participating simultaneously in the partial delimitation of one of the terminal chambers (7, 7′) and with that of the median or central chamber (6′), and the calibrated flow means (9, 9′) consist of through holes that are made in said common wall portions (12, 12′) and that have specified shapes and sizes, based on the desired sequencing for the inflation and the deflation of the chambers (7, 6′, 7′) determining the amplitude and/or the speed of the translational movement of the elongated object (2) in question.

4. The pneumatic device according to claim 1, wherein the calibration of each of the respective flow means (9, 9′) between each of the terminal chambers (5, 5′) and the median or central chamber (6), which is optionally different for each means (9, 9′), is adjusted in such a way that, by passive monitoring of the circulation of the gaseous fluid that is injected via the single feed line (10, 10′), the two terminal chambers (5 and 5′) can be under an operational pressure that brings about an effective double clamping of the object (2) that is to be moved when the median or central chamber (6) is under operational pressure and in its deformed state.

5. The pneumatic device according to claim 1, wherein the median or central part or segment (6) of the hollow body (1′) is at least partially formed or covered by a layer (13) of an auxetic material, i.e., with a negative Poisson's ratio.

6. The pneumatic device according to claim 1, wherein the multi-chamber hollow body (1′) with its three outputs or segments (5, 5′) is entirely made of polymer material(s), preferably both as a one-part piece or made integrally, for example by three-dimensional printing, with the deformable membranes (8, 8′) of the jaws (5, 5′) optionally being connected by over-molding of a specific distinct polymer material.

7. The pneumatic device according to claim 1, wherein a conduit (10, 10′) that is able and is designed to form selectively a feed line or a drain line is connected to each of the chambers (7, 7′) of the end parts or segments (5, 5′).

8. The pneumatic device according to claim 1, wherein the median or central part or segment (6) defines an annular or toroidal chamber (6′) and extends peripherally, and externally radially, around the two end parts or segments (5, 5′) to produce a multi-chamber hollow body (1′) of disk-like shape and it connects these parts or segments (5, 5′) to one another by an elastically deformable bond in the axial direction (DA), with the two end parts or segments (5, 5′) being in mutual contact in said axial direction (DA) in the deflated state of the median or central part or segment (6) and the two end parts or segments (5, 5′) being located at a distance from one another in the inflated state of said median or central part or segment (6), under a pressure that is sufficient to bring about a deformation of the latter.

9. The pneumatic device according to claim 8, wherein the median or central part or segment (6) of the multi-chamber hollow body (1′) consists of, on the one hand, a portion of the internal tubular wall (14) that can be deformed elastically in the axial direction (DA) and that connects the two end parts or segments (5, 5′) between them, and, on the other hand, an outside wall portion with a U-shaped cross-section (15), for rotation around the axial direction (DA) and that forms—by additionally cooperating with the portion of the internal tubular wall (14)—the annular or toroidal median or central chamber (6′) of the hollow body (1′), with the two wings (15′) of the U also being connected by their free ends to the two end parts or segments (5, 5′) and being elastically deformable by bending toward the outside of the U in the axial direction (DA), under the action of an adequate overpressure in the central or median chamber (6′).

10. The pneumatic device according to claim 9, wherein the areas (15″) of the outside wall portion with a U-shaped cross-section (15) that correspond to the wings (15′) of said U are formed by or covered with, preferably on their internal face, a layer (13) made of auxetic material.

11. The pneumatic device according to claim 1, wherein the median or central part or segment (6) comprises a cylindrical tubular chamber (6′) that extends in the axial direction (DA) and the rotational direction around the latter, with said chamber (6′) comprising an elastically deformable outside wall (16) and being in calibrated fluid communication with the chambers (7, 7′) of the end parts or segments (5, 5′), wherein said wall (16) is covered on the outside by a cylindrical structure with an auxetic property (16′), which is formed by a gridded network with a repetitive pattern of elementary formations (17) and which rests on and is integral with two opposite disk-like stops (17′), also connected to the above-mentioned deformable wall (16), and which forms common wall portions (12, 12′) with—or connected to—end parts or segments (5, 5′), with the above-mentioned constituent components of the median part or segment (6) being mutually arranged in such a way that the operational pressurization of the median or central chamber (6′) brings about—by action of the wall (16) on the network (16′)—a mutual spacing of the disk-like stops (17′) in the axial direction (DA), and therefore end parts or segments (5, 5′) that are respectively integral with said stops (17′).

12. The pneumatic device according to claim 11, wherein the elementary formations (17) together constitute a network (16′) with an inverted honeycomb pattern.

13. The pneumatic device according to claim 11, wherein at rest, the auxetic network (16′) is twisted around the axial direction (DA), with an operational pressurization of said network bringing about a relative translational movement and a relative rotational movement between the two end parts or segments (5 and 5′).

14. The pneumatic device according to claim 1, wherein the end parts or segments (5, 5′) and the median or central part or segment (6) are aligned with one another and the elongated object (2) traverses all of them, with the axial direction (DA) of the hollow body (1′) and the axis of translation of the elongated object (2) being combined.

15. The pneumatic device according to claim 1, wherein the end parts or segments (5, 5′) are aligned with one another and the elongated object (2) traverses both of them, with the axis of translation of the elongated object (2) being combined with the axis of alignment of said end parts or segments (5, 5′) corresponding to the axial direction (DA), the median or central part or segment (6) being offset laterally or radially in relation to this direction (DA) and the elongated object (2) not traversing it.

16. The pneumatic device according to claim 1, wherein the median or central part or segment (6) comprises three contiguous functional chambers or parts (22, 22′, 23), namely, on the one hand, two chambers (22, 22′) that are able and are designed to ensure a function of movement under the action of a deformation of at least one portion of their wall resulting from a modification of their internal pressure, and each coupled respectively to one of the end parts or segments (5, 5′) that form inflatable jaws, with which they are respectively in fluid communication by a calibrated flow means (9, 9′), and, on the other hand, a median functional part or chamber (23) that forms jaws, located between the two above-mentioned functional chambers or parts (22 and 22′) and in fluid communication by a respective calibrated flow means (24, 24′) with each of them, with all of the five parts (5, 5′, 22, 22′, 23) forming a cylindrical structure.

17. The pneumatic device according to claim 16, wherein a first of the two functional chambers (22) is configured to ensure a rotational movement around the axial direction (AD) of the elongated element (2) that is clamped by one (5) of the two end jaws (5, 5′) and the second of the two functional chambers (22′) is configured to ensure a translational movement in the axial direction (AD) of the elongated object (2) that is clamped by the median jaw (23) and the other end jaw (5′).

18. The pneumatic device according to claim 1, further comprising at least one adjustment means (25) of at least one calibrated flow means (9, 9′, 24, 24′), in particular its passage section, with such an adjustment means (25) preferably being combined with each flow means (9, 9′, 24, 24′).

19. The pneumatic device according to claim 18, wherein the or each adjustment means (25), with manual or motorized actuation, comprises a movable variable sealing element of the calibrated opening (9, 9′, 24, 24′) that forms the flow means in question.

20. The pneumatic device according to claim 1 further comprising a sensor means (26), connected to or integrated into the median or central part or segment (6) and is able and designed to deliver a signal based on the elongation of this part or this segment in the axial direction (AD) and/or around the latter.

21. The pneumatic device according to claim 1 wherein the elastically deformable internal membranes (8, 8′, 23′) of the parts or segments (5, 5′, 23) that form jaws, defining a tube and coming into contact with the elongated object (2), comprise protruding formations (28) or surface texturing ensuring a flexible centered hold of the elongated object (2) in the deflated and retracted state of said jaws, such as, for example, lips.

22. The pneumatic device according to claim 1 wherein the wall of at least one of the parts or segments (5, 5′, 23) that form jaws, preferably the walls of all of these parts, comprise(s) elastically deformable portions (29) in the inflated, active or clamped state of said jaw(s), providing a radial and/or axial structural compliance that is at least limited to the elongated object (2) that is grasped by this (these) jaw(s).

23. The pneumatic device according to claim 1 further comprising a solenoid valve (27) for control of the injection of fluid, structurally coupled to one (5) of the end parts or segments that form jaws (5, 5′).

24. The pneumatic device according to claim 1, wherein the
elongated object (2) consists of a surgical instrument or tool of oblong shape and is designed to traverse an opening or a tubular passage.

25. The pneumatic device according to claim 1 wherein the elongated object (2) consists of a piece, a tool or an instrument that is designed to be inserted into a recessed opening or that traverses a second or other piece.

26. A system for controlled sequential holding and moving of an elongated object, such as a surgical or medical instrument or tool of oblong shape, designed to be inserted into the body of a patient, said system comprising, on the one hand, at least one pneumatic holding and moving device carried by a specific support or made integral with the patient, and placed in direct contact with the skin of the latter, on the other hand, at least one pressurized gaseous fluid source, and, finally, a man-machine control interface, optionally being part of a medical installation,
wherein said at least one pneumatic holding and moving device consists of a device (1) according to claim 1, with its hollow body (1′) being held at its front-end part (5).

27. An automated system for insertion and optionally assembly or mounting, by translational motion, optionally combined with a rotational motion of an elongated object, such as a structural piece, tool or instrument, in a passage, an opening or a receiving housing adapted for another object, piece, part, element or the like, the system comprising at least one pneumatic device (1) for guided holding and moving according to claim 1, with said device (1) being mounted on an optionally movable support means, such as a robot arm or a collaborative robot, with said device (1) being an integral part, if necessary, of said robot arm or robot.

28. The system according to claim 26, further comprising at least two pneumatic holding and moving devices (1), arranged in an aligned manner according to their axial direction (DA) and each traversed by the elongated object (2) to hold and move automatically on command, with said devices (1) being fed with pressurized fluid in a coordinated manner.

29. A method for inserting an elongated instrument or tool into the body of a subject by means of a system according to claim 26, comprising installing at least one pneumatic holding and moving device (1) by aligning its axial direction (DA) with an inlet opening made in advance in the skin (19′) of the subject (19), then installing the instrument or the tool in the axial passage (4) of the hollow body (1′) of the device (1) up to a predetermined insertion position, then gradually and sequentially depressing said tool or instrument (2) by carrying out successively, for each elementary step of movement, a pressurized fluid injection phase and a phase for evacuating said fluid, with the injection phase being interrupted when the rear terminal chamber (5′) of the multi-chamber hollow body (1′) has reached an internal pressure that corresponds to its operational pressure, and repeating the cycle of the two above-mentioned phases as many times as necessary to reach the translational movement amplitude, and even the degree of depression, that is desired.

30. The method for inserting an elongated object (2), such as a structural piece, tool or instrument, into an opening or a passage of a piece by means of the system according to claim 27, the method comprising installing at least one pneumatic holding and moving device (1) by aligning its axial direction (DA) with the inlet opening of the opening or the passage in question, with the elongated object (2) being in place in the axial passage (4) of the hollow body (1′) of the device (1), then gradually and sequentially depressing said object (2) by successively carrying out, for each elementary step of movement, a pressurized fluid injection phase and a phase for evacuating said fluid, with the injection phase being interrupted when the rear terminal chamber (5′) of the multi-chamber hollow body (1′) has reached an internal pressure that corresponds to its operational pressure, and repeating the cycle of the two above-mentioned phases as many times as necessary to reach the amplitude of the translational movement, and even the degree of depression, that is desired.

31. An insertion method according to claim 29, wherein the injection phase of the next cycle begins while the evacuation phase of the cycle currently under way has yet to be completed.
